(19) United States
(12) Patent Application Publication
(10) Pub. No.: US 2018/0000450 A1
(43) Pub. Date: Jan. 4, 2018

(54) MEDICAL IMAGE DIAGNOSIS APPARATUS AND BIOLOGICAL SIGNAL MEASURING APPARATUS

(71) Applicant: Toshiba Medical Systems Corporation, Otawara-shi (JP)

(72) Inventor: Masaharu TSUYUKI, Nasushiobara (JP)

(73) Assignee: Toshiba Medical Systems Corporation, Otawara-shi (JP)

(21) Appl. No.: 15/637,301

(22) Filed: Jun. 29, 2017

(30) Foreign Application Priority Data
Jun. 29, 2016 (JP) 2016-128779
Jun. 27, 2017 (JP) 2017-124992

(51) Int. Cl. A61B 8/00; A61B 5/0402; A61B 8/14; A61B 8/08

(52) U.S. Cl. CPC A61B 8/4416; A61B 8/543; A61B 5/0402; A61B 8/14; A61B 8/5223

(57) ABSTRACT

According to one embodiment, a medical image diagnosis apparatus includes an acquisition unit and a control unit. The acquisition unit acquires information related to the measurement environment of a biological signal of a subject. The control unit controls operation for examining the subject based on the information related to the measurement environment.

CROSS-REFERENCE TO RELATED APPLICATION(S)

This application is based upon and claims the benefit of priority from Japanese Patent Application No. 2016-128779, filed Jun. 29, 2016; No. 2017-124992, filed Jun. 27, 2017; the entire contents of (all of) which are incorporated herein by reference.

### FIELD

Embodiments described herein relate generally to a medical image diagnosis apparatus and a biological signal measuring apparatus.

### BACKGROUND

A medical image diagnosis apparatus such as an X-ray computed tomography (CT) apparatus or a magnetic resonance imaging (MRI) apparatus generates CT images and MR images of a subject. These medical image diagnosis apparatuses capture the images by, for example, an imaging method called synchronous imaging.

For example, having received a biological signal of a subject, an X-ray CT apparatus irradiates X-rays at a predetermined phase of the biological signal. Electrocardiographic synchronous imaging and respiration synchronous imaging are known as examples of the synchronous imaging.

In the electrocardiographic synchronous imaging, an electrocardiogram signal is received from an electrocardiograph as a biological signal of a subject, and X-rays are irradiated at a predetermined cardiac phase of the electrocardiogram signal. In the respiration synchronous imaging, a respiratory signal is received from a breathing sensor as a biological signal of a subject, and X-rays are irradiated at a predetermined respiratory phase of the respiratory signal. Similarly, the MRI apparatus captures an MR image of a subject in synchronization with an electrocardiogram signal or a respiratory signal.

In the synchronous imaging, an image is sometimes captured without sufficient measurement of biological signals, and accordingly the synchronous imaging may not be successful. For example, in the electrocardiographic synchronous imaging, there are cases in which the electrode of the electrocardiograph is not properly connected to a subject, and an electrocardiogram signal measured is weak. This results in unsuccessful synchronous imaging. Unsuccessful synchronous imaging may also be caused by noise generated in the biological signal due to electrical charging of a patient. In the respiration synchronous imaging, an optical breathing sensor is sometimes used. However, in an environment too bright or too dark, the breathing sensor may not measure respiratory signals sufficient for the synchronous imaging, which may result in unsuccessful synchronous imaging.

### BRIEF DESCRIPTION OF THE DRAWINGS

FIG. 1 is a block diagram illustrating a configuration of a medical image diagnosis system according to a first embodiment;

FIG. 2 is a flowchart illustrating the operation of the medical image diagnosis system according to the first embodiment;

FIG. 3 is a flowchart illustrating the operation of the medical image diagnosis system according to the first embodiment;

FIG. 4 is a block diagram illustrating a configuration of a medical image diagnosis system according to a second embodiment; and

FIG. 5 is a flowchart illustrating the operation of the medical image diagnosis system according to the second embodiment.

### DETAILED DESCRIPTION

In general, according to one embodiment, a medical image diagnosis apparatus includes an acquisition unit and a control unit. The acquisition unit acquires information related to the measurement environment of a biological signal of a subject. The control unit controls operation for examining the subject based on the information related to the measurement environment.

In the following, a medical image diagnosis apparatus and a biological signal measuring apparatus according to embodiments will be described with reference to the drawings.

### First Embodiment

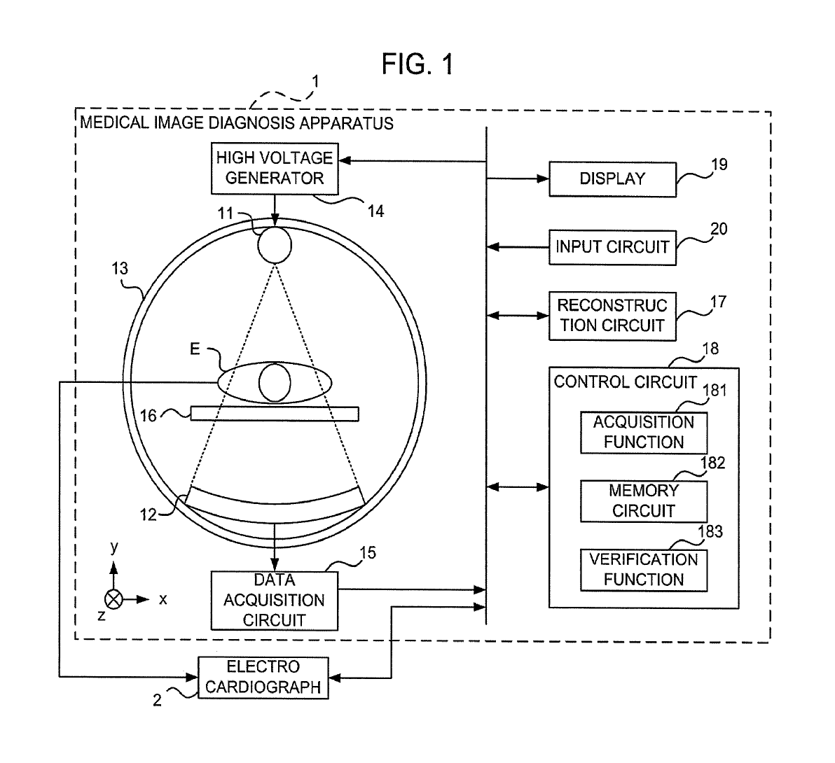

FIG. 1 is a block diagram illustrating a configuration of a medical image diagnosis system according to a first embodiment. The medical image diagnosis system of this embodiment includes a medical image diagnosis apparatus 1 and an electrocardiograph 2. For the sake of explanation, an example will be described in which the medical image diagnosis apparatus 1 is an X-ray CT apparatus. The medical image diagnosis apparatus 1 receives a biological signal of a subject E and performs synchronous imaging. The medical image diagnosis apparatus 1 includes an X-ray tube 11, an X-ray detector 12, a rotator 13, a high voltage generator 14, a data acquisition circuit 15, a bed 16, a reconstruction circuit 17, a control circuit 18, a display 19, and an input circuit 20.

In the embodiment illustrated in FIG. 1, each processing function performed by the constituent elements, the control circuit 18, an acquisition function 181, and a verification function 183 is stored in a memory circuit 182 in the form of a program executable by a computer. The control circuit 18 is a processor that reads each program from the memory circuit 182 and executes it to thereby realize a function corresponding to the program. In other words, having read programs, the control circuit 18 has the functions illustrated in the control circuit 18 in FIG. 1. Although FIG. 1 illustrates a single control circuit (18) that realizes the processing functions of the control circuit 18, the acquisition function 181 and the verification function 183, a plurality of independent processors may be combined to form a processing circuit, and each of the processors may execute a program to realize its function.

The term “processor” as used herein refers to a circuit such as, for example, a central processing unit (CPU), a graphics processing unit (GPU), an application specific integrated circuit (ASIC), a programmable logic device including a simple programmable logic device (SPLD) and a complex programmable logic device (CPLD), a field programmable gate array (FPGA), or the like. The processor reads programs out of the memory circuit 182 and executes them to thereby realize the functions. The programs need not necessarily be stored in the memory circuit 182, but may be directly incorporated in the circuit of the processor. In this case, the processor realizes the functions by reading and executing the programs incorporated in the circuit. Each processor of the embodiment need not necessarily be configured as a single circuit. A plurality of independent circuits may be combined to form a single processor for implementing the functions. Besides, a plurality of constituent elements in FIG. 1 may be integrated into one processor to realize the functions.

### [Basic Configuration]

Described below is the basic configuration of the medical image diagnosis system. The X-ray tube 11 generates X-rays. The X-rays generated are irradiated to the subject E. The X-ray detector 12 detects the X-rays that have passed through the subject E. The X-ray detector 12 is formed of a plurality of general X-ray detection elements.

The rotator 13 rotates the X-ray tube 11 and the X-ray detector 12 around the subject E. The rotator 13 is a member that supports the X-ray tube 11 and the X-ray detector 12 at such positions that they face each other with the subject E between them. The rotator 13 has an opening that passes therethrough in the slice direction. The bed 16 on which the subject E is placed is inserted into the opening. The high voltage generator 14 applies a high voltage to the X-ray tube 11. The X-ray tube 11 generates X-rays based on the high voltage applied.

The data acquisition circuit 15 acquires X-ray detection data from the X-ray detector 12. The data acquisition circuit 15 amplifies the detection data, and performs analog to digital (A/D) conversion thereof. The data acquisition circuit 15 outputs the X-ray detection data to the reconstruction circuit 17.

The bed 16 is moved in the body axis direction (z-axis direction) of the subject E while the subject E is placed thereon. The bed 16 may be moved in the vertical direction (y-axis direction). The reconstruction circuit 17 applies a reconstruction process to the X-ray detection data received from the data acquisition circuit 15 to generate a CT image.

The control circuit 18 controls each part of the medical image diagnosis apparatus 1. The control circuit 18 stores in advance a computer program for controlling each part of the medical image diagnosis apparatus 1. The control circuit 18 controls each part of the medical image diagnosis apparatus 1 by executing the computer program. Details will be described later.

The display 19 is formed of a display device such as a liquid crystal display (LCD). The display 19 is an example of the notification unit in the claims. The input circuit 20 includes operation devices such as a keyboard, a mouse, a track ball and a joystick.

The electrocardiograph 2 measures an electrocardiogram signal of the subject E and outputs it to the control circuit 18. The electrocardiograph 2 is an example of the biological signal measuring apparatus in the claims. The electrocardiograph 2 has an electrode. An operator such as a doctor or a technician sticks adheres the electrode to the subject E such that the electrocardiograph 2 is prepared to measure electrocardiogram signal. The electrocardiogram signal is an example of the biological signal in the claims. The electrocardiograph 2 is communicably connected to the medical image diagnosis apparatus 1. Note that the electrocardiograph 2 may be mounted inside the medical image diagnosis apparatus 1.

### [Control Configuration]

The control configuration of the medical image diagnosis system will be described. The control circuit 18 acquires information related to the measurement environment of the electrocardiogram signal measured by the electrocardiograph 2 (the acquisition function 181). For example, the electrocardiograph 2 measures impedance related to the measurement of the electrocardiogram signal, and outputs impedance information indicating the impedance to the control circuit 18. Thus, the control circuit 18 acquires the impedance information. The timing at which the control circuit 18 acquires the impedance information may be manually specified by the operator, or it may be set in advance such that the information can be acquired automatically at a predetermined timing. The impedance information is an example of the information related to the measurement environment. A general method may be used to measure the impedance.

The control circuit 18 has the memory circuit 182. The memory circuit 182 is an example of the storage in the claims. The memory circuit 182 stores in advance an impedance threshold which is a threshold value related to the impedance. The impedance threshold is provided in advance by the operator such as a doctor or a technician.

The control circuit 18 is configured to be able to control the operation for examining the subject E based on the impedance information when having acquired the impedance information. For example, the control circuit 18 compares the impedance information acquired from the electrocardiograph 2 with the impedance threshold stored in the memory circuit 182 (the verification function 183). The verification function 183 is an example of the verification unit in the claims. The control circuit 18 is configured to be able to stop the progress of the operation for examining the subject E based on the result of the verification. For example, the control circuit 18 stops the progress of the operation when the impedance indicated by the impedance information is higher than the impedance threshold.

When stopping the progress of the operation for examining the subject E, the control circuit 18 controls the display 19 to provide information that recommends the operator to take measures for reducing the impedance. For example, the control circuit 18 displays on the display 19 text information recommending the replacement of the electrode or the use of a medical gel. The information displayed on the display 19 is not limited to text information, but may be predetermined image information or the like. Thereby, the operator can take measures to reduce the impedance. The information displayed at this time is set in advance.

When synchronous imaging is performed in a state where the impedance is high, the synchronous imaging may not succeed in some cases since the electrocardiogram signal measured is weak. By stopping the progress of the operation and recommending measures to reduce the impedance when the impedance is high, the synchronous imaging can be performed while the electrocardiogram signal is measured successfully.

Besides, the electrocardiograph 2 measures the charge voltage of the electrode, and outputs charge information indicating the charge voltage to the control circuit 18. The timing at which the control circuit 18 acquires the charge information may be manually specified by the operator, or it may be set in advance such that the information can be acquired automatically at a predetermined timing. The charge information is an example of the information related to the measurement environment. A general method may be used to measure the charge information. The memory circuit 182 stores in advance a charge threshold which is a threshold value related to the charge voltage. The charge threshold is provided in advance by the operator such as a doctor or a technician.

The control circuit 18 is configured to be able to control the operation for examining the subject E based on the charge information when having acquired the charge information. For example, the control circuit 18 compares the charge information acquired from the electrocardiograph 2 with the charge threshold stored in the memory circuit 182. The control circuit 18 is configured to be able to stop the progress of the operation for examining the subject E based on the result of the verification. For example, when the charge voltage indicated by the charge information is higher than the charge threshold, the control circuit 18 stops the progress of the operation.

When stopping the progress of the operation for examining the subject E, the control circuit 18 controls the display 19 to provide information that recommends the operator to take measures for reducing the charge voltage. For example, the control circuit 18 displays on the display 19 text information recommending an increase in humidity around the subject E. The information displayed on the display 19 is not limited to text information, but may be predetermined image information or the like. Thereby, the operator can take measures to reduce the charge voltage. The information displayed at this time is set in advance.

The patient may be charged with static electricity. Besides, it is known that X-rays have a property of reducing static electricity. When synchronous imaging is performed in a state in which the charge voltage is high, the measurement of the electrocardiogram signal and the irradiation of X-rays are simultaneously performed in parallel. The reduction of static electricity that has already been charged corresponds to charge transfer. The charge transfer generates a current. When synchronous imaging is performed in a state in which the charge voltage is high, this current becomes noise to the electrocardiogram signal, and the synchronous imaging may not be successful. By recommending measures to reduce the charge voltage before synchronous imaging is performed, the synchronous imaging can be performed while the electrocardiogram signal is measured successfully.

In addition, the electrocardiograph 2 measures the cycle or amplitude of the electrocardiogram signal or both of them, and outputs cycle information indicating the cycle, amplitude information indicating the amplitude, or both to the control circuit 18. The timing at which the control circuit 18 acquires the cycle information and the amplitude information may be manually specified by the operator, or it may be set in advance such that the information can be acquired automatically at a predetermined timing. The cycle information and the amplitude information are examples of the information related to the measurement environment. A general method may be used to measure the cycle and the amplitude. The memory circuit 182 stores in advance a cycle threshold which is a threshold value related to the cycle or an amplitude threshold which is a threshold value related to the amplitude, or both. For the cycle threshold and the amplitude threshold, an upper limit value and a lower limit value are provided in advance by the operator such as a doctor or a technician.

When acquiring the cycle information, the amplitude information, or both of them as the information related to the measurement environment, the control circuit 18 can control the operation for examining the subject E based on the quality information. For example, the control circuit 18 compares the cycle information or the amplitude information acquired from the electrocardiograph 2 or both of them with the cycle threshold or the amplitude threshold stored in the memory circuit 182 or both.

The control circuit 18 is configured to be able to stop the progress of the operation for examining the subject E based on the result of the verification. For example, when the cycle indicated by the cycle information does not fall between the upper limit value and the lower limit value of the cycle threshold, the control circuit 18 stops the progress of the operation.

When stopping the progress of the operation for examining the subject E, the control circuit 18 controls the display 19 to provide information that recommends the operator to take measures for improving the cycle. The improvement of the cycle means that the cycle of the electrocardiogram signal falls between the upper limit value and the lower limit value of the cycle threshold. For example, the control circuit 18 displays on the display 19 text information recommending the administration of a medicine for heart rate adjustment to the subject E. The information displayed on the display 19 is not limited to text information, but may be predetermined image information or the like. Thereby, the operator can take measures to improve the cycle. The information displayed at this time is set in advance.

It is known that there is a cardiac cycle suitable for synchronous imaging. If synchronous imaging is performed when the cardiac cycle is not suitable, an image cannot be captured with good timing at every predetermined cardiac phase, and the synchronous imaging may not be successful. By recommending measures to improve the cycle before the synchronous imaging is performed, the synchronous imaging can be performed while the electrocardiogram signal is measured successfully.

Further, for example, when the amplitude indicated by the amplitude information does not fall between the upper limit value and the lower limit value of the amplitude threshold, the control circuit 18 stops the progress of the operation for examining the subject E. When stopping the progress of the operation for examining the subject E, the control circuit 18 controls the display 19 to provide information that recommends the operator to take measures for improving the amplitude. The improvement of the amplitude means that the amplitude of the electrocardiogram signal falls between the upper limit value and the lower limit value of the amplitude threshold. For example, the control circuit 18 displays on the display 19 text information recommending the gain adjustment of the electrocardiograph 2. The information displayed on the display 19 is not limited to text information, but may be predetermined image information or the like. Thereby, the operator can take measures to improve the amplitude. The information displayed at this time is set in advance. When stopping the progress of the operation for examining the subject E, the control circuit 18 may automatically control the electrocardiograph 2 to adjust the gain. The automatic adjustment program may be set in advance by a doctor, a technician or the like.

It is known that there is an amplitude suitable for synchronous imaging. If synchronous imaging is performed when the amplitude is not suitable, an image cannot be captured with good timing at every predetermined cardiac phase, and the synchronous imaging may not be successful. By recommending measures to improve the amplitude before the synchronous imaging is performed, the synchronous imaging can be performed while the electrocardiogram signal is measured successfully.

The electrocardiograph 2 may measure the S/N ratio of the electrocardiogram signal and output S/N ratio information indicating the S/N ratio to the control circuit 18. The S/N ratio information is an example of the information related to the measurement environment. A general method may be used to measure the S/N ratio. The memory circuit 182 stores in advance an S/N ratio threshold which is a threshold value related to the S/N ratio. The S/N ratio threshold is provided in advance by the operator such as a doctor or a technician.

The control circuit 18 is configured to be able to control the operation for examining the subject E based on the S/N ratio information when having acquired the S/N ratio information. For example, the control circuit 18 compares the S/N ratio information acquired from the electrocardiograph 2 with the S/N ratio threshold stored in the memory circuit 182.

The control circuit 18 is configured to be able to stop the progress of the operation for examining the subject E based on the result of the verification. For example, when the S/N ratio indicated by the S/N ratio information is smaller than the S/N ratio threshold, the control circuit 18 stops the progress of the operation.

When stopping the progress of the operation for examining the subject E, the control circuit 18 controls the display 19 to provide information that recommends the operator to take measures for improving the S/N ratio. The improvement of the S/N ratio means that the S/N ratio of the electrocardiogram signal becomes equal to or higher than the S/N ratio threshold. For example, the control circuit 18 displays on the display 19 text information recommending the adjustment of parameters such as the gain of the electrocardiograph 2 and the replacement of the electrode. The information displayed on the display 19 is not limited to text information, but may be predetermined image information or the like. Thereby, the operator can take measures to improve the S/N ratio. When stopping the progress of the operation for examining the subject E, the control circuit 18 may automatically control the electrocardiograph 2 to adjust the S/N ratio. The automatic adjustment program may be set in advance by a doctor, a technician or the like.

If synchronous imaging is performed when the S/N ratio is low, an image cannot be captured with good timing at every predetermined cardiac phase, and the synchronous imaging may not be successful. By recommending measures to improve the S/N ratio before the synchronous imaging is performed, the synchronous imaging can be performed while the electrocardiogram signal is measured successfully.

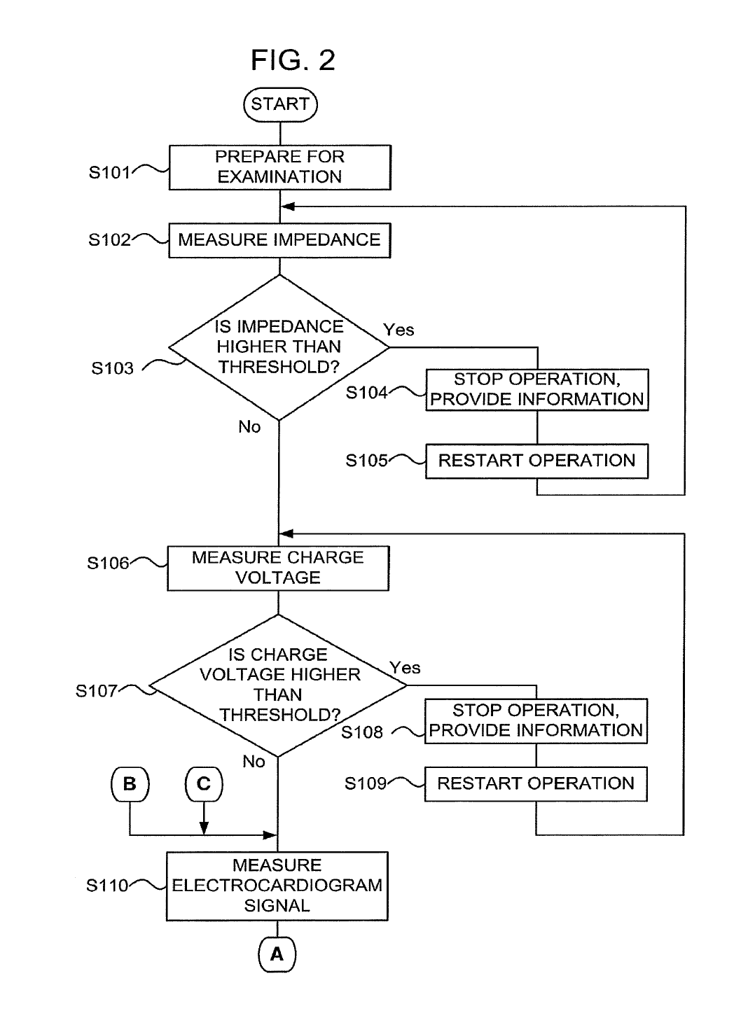

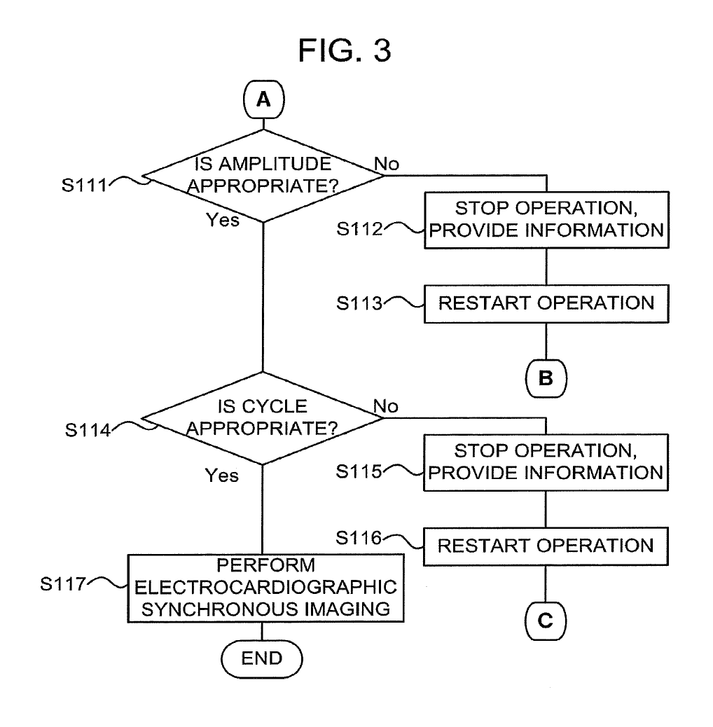

FIGS. 2 and 3 are flowcharts illustrating the operation of the medical image diagnosis system according to the first embodiment.

Step S101: The subject E is placed on the bed 16, and the electrode of the electrocardiograph 2 is adhered to the subject E by the operator.

Step S102: The electrocardiograph 2 measures impedance related to the measurement of an electrocardiogram signal, and outputs impedance information indicating the impedance to the control circuit 18. Thus, the control circuit 18 acquires the impedance information.

Step S103: The control circuit 18 compares the impedance information acquired from the electrocardiograph 2 with the impedance threshold stored in the memory circuit 182. When the impedance indicated by the impedance information is higher than the impedance threshold (Yes in step S103), the process proceeds to step S104. When the impedance indicated by the impedance information is not higher than the impedance threshold (No in step S103), the process proceeds to step S106.

Step S104: The control circuit 18 stops the progress of the operation for examining the subject E. Then, the control circuit 18 controls the display 19 to provide information recommending measures for reducing the impedance.

Step S105: The operator operates the input circuit 20 to provide an input to restart the operation for examining the subject E. Upon receipt of the input, the control circuit 18 restarts the operation for examining the subject E, and the process returns to step S102. Incidentally, the operation for examining the subject E may be set in advance so as to be restarted automatically.

Step S106: The electrocardiograph 2 measures the charge voltage of the electrode, and outputs charge information indicating the charge voltage to the control circuit 18. Thus, the control circuit 18 acquires charge information.

Step S107: The control circuit 18 compares the charge information acquired from the electrocardiograph 2 with the charge threshold stored in the memory circuit 182. When the charge voltage indicated by the charge information is higher than the charge threshold (Yes in step S107), the process proceeds to step S108. When the charge voltage indicated by the charge information is not higher than the charge threshold (No in step S107), the process proceeds to step S110.

Step S108: The control circuit 18 stops the progress of the operation for examining the subject E. Then, the control circuit 18 controls the display 19 to provide information recommending measures to reduce the charge voltage.

Step S109: The operator operates the input circuit 20 to provide an input to restart the operation for examining the subject E. Upon receipt of the input, the control circuit 18 restarts the operation for examining the subject E, and the process returns to step S106. Incidentally, the operation for examining the subject E may be set in advance so as to be restarted automatically.

Step S110: The electrocardiograph 2 measures an electrocardiogram signal of the subject E. The electrocardiograph 2 outputs cycle information indicating the cycle of the electrocardiogram signal and amplitude information indicating the amplitude to the control circuit 18. Thus, the control circuit 18 acquires the cycle information and the amplitude information.

Step S111: The control circuit 18 compares the amplitude information acquired from the electrocardiograph 2 with the amplitude threshold stored in the memory circuit 182. When the amplitude indicated by the amplitude information does not fall between the upper limit value and the lower limit value of the amplitude threshold (No in step S111), the process proceeds to step S112. When the amplitude indicated by the amplitude information falls between the upper limit value and the lower limit value of the amplitude threshold (Yes in step S111), the process proceeds to step S114.

Step S112: The control circuit 18 stops the progress of the operation for examining the subject E. The control circuit 18 controls the display 19 to provide information recommending measures for improving the amplitude.

Step S113: The operator operates the input circuit 20 to provide an input to restart the operation for examining the subject E. Upon receipt of the input, the control circuit 18 restarts the operation for examining the subject E, and the process returns to step S110. Incidentally, the operation for examining the subject E may be set in advance so as to be restarted automatically.

Step S114. The control circuit 18 compares the cycle information acquired from the electrocardiograph 2 with the cycle threshold stored in the memory circuit 182. When the cycle indicated by the cycle information does not fall between the upper limit value and the lower limit value of the cycle threshold (No in step S114), the process proceeds to step S115. When the cycle indicated by the cycle information falls between the upper limit value and the lower limit value of the cycle threshold (Yes in step S114), the process proceeds to step S117.

Step S115: The control circuit 18 stops the progress of the operation for examining the subject E. Then, the control circuit 18 controls the display 19 to provide information recommending measures to improve the cycle.

Step S116: The operator operates the input circuit 20 to provide an input to restart the operation for examining the subject E. Upon receipt of the input, the control circuit 18 restarts the operation for examining the subject E, and the process returns to step S110. Incidentally, the operation for examining the subject E may be set in advance so as to be restarted automatically.

Step S117: The medical image diagnosis apparatus 1 performs electrocardiographic synchronous imaging each time when a predetermined cardiac phase arrives while receiving the electrocardiogram signal of the subject E from the electrocardiograph 2.

Note that the order of the process group of steps S102 to S105 and the process group of steps S106 to S109 may be arbitrarily changed.

According to this embodiment, information related to the measurement environment of the electrocardiogram signal is acquired and is compared with a threshold stored in advance before electrocardiographic synchronous imaging is performed. Thereby, the synchronous imaging can be performed while the electrocardiogram signal is measured successfully.

### Second Embodiment

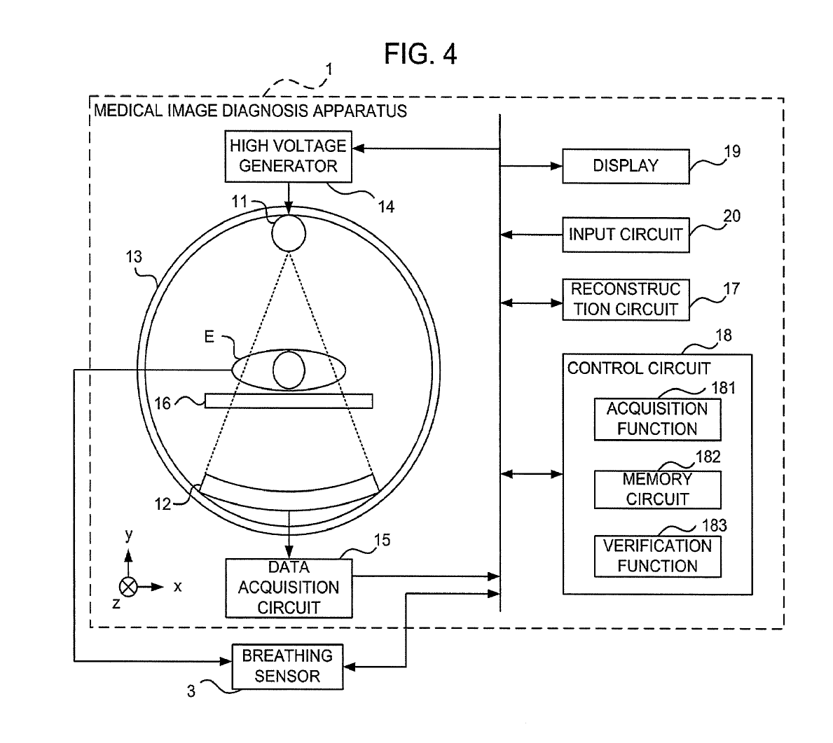

FIG. 4 is a block diagram illustrating a configuration of a medical image diagnosis system according to a second embodiment. The medical image diagnosis system of this embodiment includes the medical image diagnosis apparatus 1 and a breathing sensor 3. In the following, differences from the first embodiment will be mainly described.

The breathing sensor 3 measures a respiratory signal of the subject E and outputs it to the control circuit 18. The breathing sensor 3 is an example of the biological signal measuring apparatus in the claims. For example, the breathing sensor 3 includes a general optical sensor, and detects the movement of the body surface (abdomen, chest, etc.) due to the respiratory motion of the subject E as a respiratory signal. In addition, the breathing sensor 3 includes a general actinometer and measures the surrounding light intensity. The breathing sensor 3 may be the one having an air flow rate detector attached to the mouth of the subject E and detecting a change in air flow rate as a respiratory signal. The operator such as a doctor or a technician prepares the breathing sensor 3 to measure the respiratory signal of the subject E. The respiratory signal is an example of the biological signal in the claims. The breathing sensor 3 is communicably connected to the medical image diagnosis apparatus 1. Note that the breathing sensor 3 may be mounted inside the medical image diagnosis apparatus 1.

The control circuit 18 acquires information related to the measurement environment of the respiratory signal measured by the breathing sensor 3 (the acquisition function 181). For example, the breathing sensor 3 measures the light amount (intensity) used for measuring the respiratory signal or the light intensity of the ambient light, and outputs light amount information indicating the light amount measured to the control circuit 18. Thus, the control circuit 18 acquires the light amount information. The timing at which the control circuit 18 acquires the light amount information may be manually specified by the operator, or it may be set in advance such that the information can be acquired automatically at a predetermined timing. The light amount information is an example of the information related to the measurement environment.

The memory circuit 182 stores in advance a light amount threshold which is a threshold value related to the light amount. The light intensity threshold is provided in advance by the operator such as a doctor or a technician.

The control circuit 18 is configured to be able to control the operation for examining the subject E based on the light amount information when having acquired the light amount information. For example, the control circuit 18 compares the light amount information acquired from the breathing sensor 3 with the light amount threshold stored in the memory circuit 182 (the verification function 183). The control circuit 18 is configured to be able to stop the progress of the operation for examining the subject E based on the result of the verification. For example, when the amount of light used for measurement is lower than the light amount threshold, the control circuit 18 stops the progress of the operation. Alternatively, the control circuit 18 stops the progress of the operation when the variation of the light amount of the ambient light is larger than the light amount threshold.

When stopping the progress of the operation for examining the subject E, the control circuit 18 controls the display 19 to provide information that recommends the operator to take measures for increasing the amount of light used for measurement, and for reducing the variation of the light amount of the ambient light. For example, the control circuit 18 displays on the display 19 text information that recommends the adjustment of lighting equipment in the examination room where the medical image diagnosis apparatus 1 is installed. The information displayed on the display 19 is not limited to text information, but may be predetermined image information or the like. Thereby, the operator can take measures to increase the amount of light used for measurement and to reduce the variation of the ambient light. The information displayed at this time is set in advance.

If synchronous imaging is performed when the amount of light used for measurement is small or when the ambient light varies greatly, the respiratory motion of the subject E cannot be sufficiently detected and the synchronous imaging may not be successful. By stopping the progress of the operation and recommending measures to increase the amount of light used for measurement and to reduce the variation of the ambient light when the amount of light used for measurement is small or when the ambient light varies greatly, the synchronous imaging can be performed while the electrocardiogram signal is measured successfully.

Besides, the breathing sensor 3 measures the cycle or amplitude of the respiratory signal or both of them, and outputs cycle information indicating the cycle, amplitude information indicating this amplitude, or both to the control circuit 18. The timing at which the control circuit 18 acquires the cycle information and the amplitude information may be manually specified by the operator, or it may be set in advance such that the information can be acquired automatically at a predetermined timing. The cycle information and the amplitude information are examples of the information related to the measurement environment. A general method may be used to measure the cycle and the amplitude. The memory circuit 182 stores in advance a cycle threshold which is a threshold value related to the cycle or an amplitude threshold which is a threshold value related to the amplitude, or both. For the cycle threshold and the amplitude threshold, an upper limit value and a lower limit value are provided in advance by the operator such as a doctor or a technician.

When acquiring the cycle information, the amplitude information, or both of them as quality information, the control circuit 18 can control the operation for examining the subject E based on the quality information. For example, the control circuit 18 compares the cycle information or the amplitude information acquired from the breathing sensor 3 or both of them with the cycle threshold or the amplitude threshold stored in the memory circuit 182 or both.

The control circuit 18 is configured to be able to stop the progress of the operation for examining the subject E based on the result of the verification. For example, when the cycle indicated by the cycle information does not fall between the upper limit value and the lower limit value of the cycle threshold, the control circuit 18 stops the progress of the operation.

When stopping the progress of the operation for examining the subject E, the control circuit 18 controls the display 19 to provide information that recommends the operator to take measures for improving the cycle. The improvement of the cycle means that the cycle of the respiratory signal falls between the upper limit value and the lower limit value of the cycle threshold. For example, the control circuit 18 displays on the display 19 text information indicating an instruction for the subject E to adjust the respiration cycle. The information displayed on the display 19 is not limited to text information, but may be predetermined image information or the like. Thereby, the operator can take measures to improve the cycle. The information displayed at this time is set in advance.

It is known that there is a respiratory cycle suitable for synchronous imaging. If synchronous imaging is performed when the respiratory cycle is not suitable, an image cannot be captured with good timing at every predetermined respiratory phase, and the synchronous imaging may not be successful. By recommending measures to improve the cycle before the synchronous imaging is performed, the synchronous imaging can be performed while the respiratory signal is measured successfully.

Further, for example, when the amplitude indicated by the amplitude information does not fall between the upper limit value and the lower limit value of the amplitude threshold, the control circuit 18 stops the progress of the operation for examining the subject E. When stopping the progress of the operation for examining the subject E, the control circuit 18 controls the display 19 to provide information that recommends the operator to take measures for improving the amplitude. The improvement of the amplitude means that the amplitude of the respiratory signal falls between the upper limit value and the lower limit value of the amplitude threshold. For example, the control circuit 18 displays on the display 19 text information recommending the gain adjustment of the breathing sensor 3. The information displayed on the display 19 is not limited to text information, but may be predetermined image information or the like. Thereby, the operator can take measures to improve the amplitude. The information displayed at this time is set in advance. When stopping the progress of the operation for examining the subject E, the control circuit 18 may automatically control the breathing sensor 3 to adjust the gain. The automatic adjustment program may be set in advance by a doctor, a technician or the like.

It is known that there is an amplitude suitable for synchronous imaging. If synchronous imaging is performed when the amplitude is not suitable, an image cannot be captured with good timing at every predetermined respiratory phase, and the synchronous imaging may not be successful. By recommending measures to improve the amplitude before the synchronous imaging is performed, the synchronous imaging can be performed while the respiratory signal is measured successfully.

The breathing sensor 3 may measure the S/N ratio of the respiratory signal and output S/N ratio information indicating the S/N ratio to the control circuit 18. The S/N ratio information is an example of the information related to the measurement environment. A general method may be used to measure the S/N ratio. The memory circuit 182 stores in advance an S/N ratio threshold which is a threshold value related to the S/N ratio. The S/N ratio threshold is provided in advance by the operator such as a doctor or a technician.

The control circuit 18 is configured to be able to control the operation for examining the subject E based on the S/N ratio information when having acquired the S/N ratio information. For example, the control circuit 18 compares the S/N ratio information acquired from the breathing sensor 3 with the S/N ratio threshold stored in the memory circuit 182.

The control circuit 18 is configured to be able to stop the progress of the operation for examining the subject E based on the result of the verification. For example, when the S/N ratio indicated by the S/N ratio information is smaller than the S/N ratio threshold, the control circuit 18 stops the progress of the operation.

When stopping the progress of the operation for examining the subject E, the control circuit 18 controls the display 19 to provide information that recommends the operator to take measures for improving the S/N ratio. The improvement of the S/N ratio means that the S/N ratio of the respiratory signal becomes equal to or higher than the S/N ratio threshold. For example, the control circuit 18 displays on the display 19 text information recommending the adjustment of parameters such as the gain of the breathing sensor 3 and the replacement of the optical sensor. The information displayed on the display 19 is not limited to text information, but may be predetermined image information or the like. Thereby, the operator can take measures to improve the S/N ratio. When stopping the progress of the operation for examining the subject E, the control circuit 18 may automatically control the breathing sensor 3 to adjust the S/N ratio. The automatic adjustment program may be set in advance by a doctor, a technician or the like.

If synchronous imaging is performed when the S/N ratio is low, an image cannot be captured with good timing at every predetermined respiratory phase, and the synchronous imaging may not be successful. By recommending measures to improve the S/N ratio before the synchronous imaging is performed, the synchronous imaging can be performed while the respiratory signal is measured successfully.

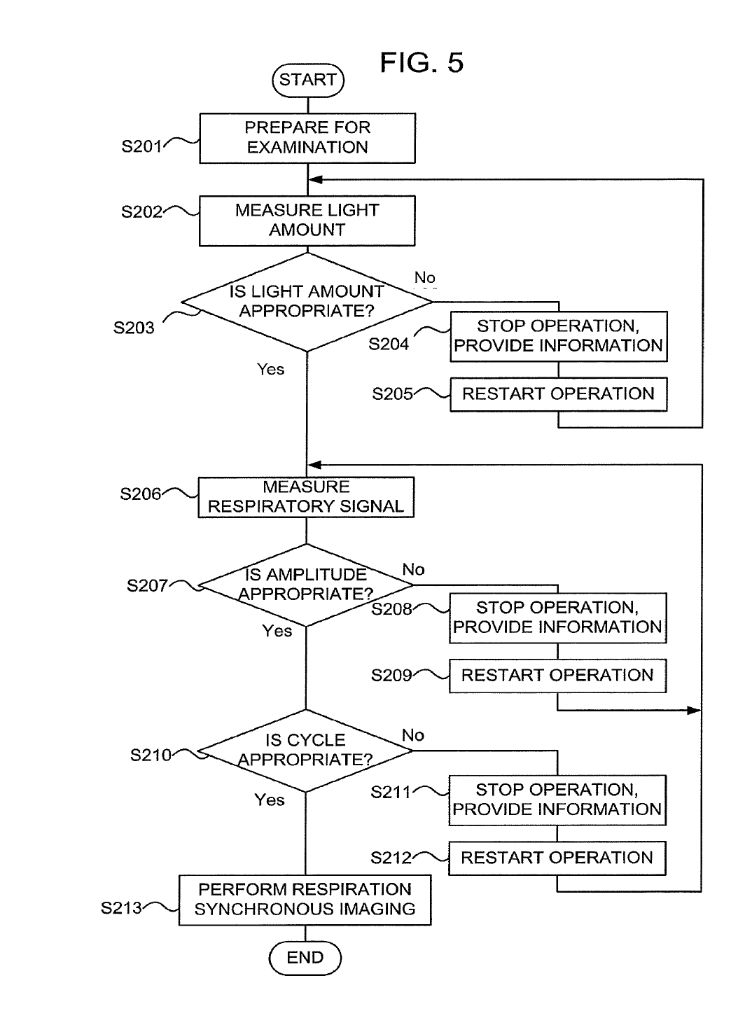

FIG. 5 is a flowchart illustrating the operation of the medical image diagnosis system according to the second embodiment.

Step S201: the subject E is placed on the bed 16, and the breathing sensor 3 is set by the operator.

Step S202: The breathing sensor 3 measures the amount of light used for measurement or the amount of ambient light, and outputs light amount information indicating the amount of light to the control circuit 18. Thus, the control circuit 18 acquires the light amount information.

Step S203: The control circuit 18 compares the light amount information acquired from the breathing sensor 3 with the light amount threshold stored in the memory circuit 182. When the light amount indicated by the light amount information is not appropriate (No in step S203), the process proceeds to step S204. When the light amount indicated by the light amount information is appropriate (Yes in step S203), the process proceeds to step S206.

Step S204: The control circuit 18 stops the progress of the operation for examining the subject E. Then, the control circuit 18 controls the display 19 to provide information recommending measures for adjusting the light amount.

Step S205: The operator operates the input circuit 20 to provide an input to restart the operation for examining the subject E. Upon receipt of the input, the control circuit 18 restarts the operation for examining the subject E, and the process returns to step S202. Incidentally, the operation for examining the subject E may be set in advance so as to be restarted automatically.

Step S206: The breathing sensor 3 measures a respiratory signal of the subject E. The breathing sensor 3 outputs cycle information indicating the cycle of the respiratory signal and amplitude information indicating the amplitude to the control circuit 18. Thus, the control circuit 18 acquires the cycle information and the amplitude information.

Step S207: The control circuit 18 compares the amplitude information acquired from the breathing sensor 3 with the amplitude threshold stored in the memory circuit 182. When the amplitude indicated by the amplitude information does not fall between the upper limit value and the lower limit value of the amplitude threshold (No in step S207), the process proceeds to step S208. When the amplitude indicated by the amplitude information falls between the upper limit value and the lower limit value of the amplitude threshold (Yes in step S207), the process proceeds to step S210.

Step S208: The control circuit 18 stops the progress of the operation for examining the subject E. The control circuit 18 controls the display 19 to provide information recommending measures for improving the amplitude.

Step S209: The operator operates the input circuit 20 to provide an input to restart the operation for examining the subject E. Upon receipt of the input, the control circuit 18 restarts the operation for examining the subject E, and the process returns to step S206. Incidentally, the operation for examining the subject E may be set in advance so as to be restarted automatically.

Step S210: The control circuit 18 compares the cycle information acquired from the breathing sensor 3 with the cycle threshold stored in the memory circuit 182. When the cycle indicated by the cycle information does not fall between the upper limit value and the lower limit value of the cycle threshold (No in step S210), the process proceeds to step S211. When the cycle indicated by the cycle information falls between the upper limit value and the lower limit value of the cycle threshold (Yes in step S210), the process proceeds to step S213.

Step S211: The control circuit 18 stops the progress of the operation for examining the subject E. Then, the control circuit 18 controls the display 19 to provide information recommending measures to improve the cycle.

Step S212: The operator operates the input circuit 20 to provide an input to restart the operation for examining the subject E. Upon receipt of the input, the control circuit 18 restarts the operation for examining the subject E, and the process returns to step S206. Incidentally, the operation for examining the subject E may be set in advance so as to be restarted automatically.

Step S213: The medical image diagnosis apparatus 1 performs respiration synchronous imaging each time when a predetermined respiratory phase arrives while receiving the respiratory signal of the subject E from the breathing sensor 3.

According to this embodiment, information related to the measurement environment of the respiratory signal is acquired and is compared with a threshold stored in advance before respiration synchronous imaging is performed. Thereby, the synchronous imaging can be performed while the respiratory signal is measured successfully.

With the medical image diagnosis apparatus and the biological signal measuring apparatus according to at least one embodiment described above, information related to the measurement environment of the biological signal is acquired and is compared with a threshold stored in advance before synchronous imaging is performed. Thereby, the synchronous imaging can be performed while the biological signal is measured successfully.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions. For example, in this specification, the medical image diagnosis apparatus is described as an X-ray CT apparatus by way of example; however, the configuration of the above embodiments may be applied to other medical image diagnosis apparatuses such as an MRI apparatus.

What is claimed is:

1. A medical image diagnosis apparatus comprising processing circuitry configured to:
acquire information related to measurement environment of a biological signal of a subject, and
control operation for examining the subject based on the information related to the measurement environment.

2. The medical image diagnosis apparatus of claim 1, wherein
the processing circuitry is communicably connected to a biological signal measuring apparatus configured to measure the biological signal, and
the processing circuitry is further configured to acquire the information related to the measurement environment from the biological signal measuring apparatus.

3. The medical image diagnosis apparatus of claim 1, further comprising a biological signal measuring apparatus configured to measure the biological signal,
wherein the processing circuitry is further configured to acquire the information related to the measurement environment from the biological signal measuring apparatus.

4. The medical image diagnosis apparatus of claim 3, wherein
the biological signal measuring apparatus is an electrocardiograph, and
when acquiring charge information indicating charge voltage of the electrocardiograph as the information related to the measurement environment, the processing circuitry controls the operation for examining the subject based on the charge information.

5. The medical image diagnosis apparatus of claim 4, wherein
the processing circuitry includes a storage configured to store a charge threshold, which is a threshold related to the charge voltage, in advance, and
the processing circuitry is further configured to:
compare the charge information with the charge threshold; and
stop the operation for examining the subject based on a result of the comparison.

6. The medical image diagnosis apparatus of claim 5, further comprising a notification unit configured to, when the operation for examining the subject is stopped, provide information that recommends measures to reduce the charge voltage.

7. The medical image diagnosis apparatus of claim 3, wherein
the biological signal measuring apparatus is an electrocardiograph, and
when acquiring impedance information indicating impedance of the electrocardiograph as the information related to the measurement environment, the processing circuitry controls the operation for examining the subject based on the impedance information.

8. The medical image diagnosis apparatus of claim 7, wherein
the processing circuitry includes a storage configured to store an impedance threshold, which is a threshold related to the impedance, in advance, and
the processing circuitry is further configured to:
compare the impedance information with the impedance threshold; and
stop the operation for examining the subject based on a result of the comparison.

9. The medical image diagnosis apparatus of claim 8, further comprising a notification unit configured to, when the operation for examining the subject is stopped, provide information that recommends measures to reduce the impedance.

10. The medical image diagnosis apparatus of claim 3, wherein
the biological signal measuring apparatus is an optical breathing sensor, and
when acquiring light amount information indicating an amount of light around the breathing sensor as the information related to the measurement environment, the processing circuitry controls the operation for examining the subject based on the light amount information.

11. The medical image diagnosis apparatus of claim 10, wherein
the processing circuitry includes a storage configured to store a light amount threshold, which is a threshold related to the light amount, in advance, and
the processing circuitry is further configured to:
compare the light amount information with the light amount threshold; and
stop the operation for examining the subject based on a result of the comparison.

12. The medical image diagnosis apparatus of claim 11, further comprising a notification unit configured to, when the operation for examining the subject is stopped, provide information that recommends measures to increase light amount used for measurement performed by the breathing sensor.

13. The medical image diagnosis apparatus of claim 11, further comprising a notification unit configured to, when the operation for examining the subject is stopped, provide information that recommends measures to reduce variation in ambient light of the breathing sensor.

14. The medical image diagnosis apparatus of claim 3, wherein
the biological signal measuring apparatus is an electrocardiograph, and
when acquiring cycle information indicating cycle of an electrocardiogram signal measured by the electrocardiograph as the biological signal or amplitude information indicating amplitude of the electrocardiogram signal as the information related to the measurement environment, the processing circuitry controls the operation for examining the subject based on the information related to the measurement environment.

15. The medical image diagnosis apparatus of claim 14, wherein
the processing circuitry includes a storage configured to store a cycle threshold, which is a threshold related to the cycle, or an amplitude threshold, which is a threshold related to the amplitude, in advance, and
the processing circuitry is further configured to:
compare the electrocardiogram signal with the cycle threshold or the amplitude threshold; and
stop the operation for examining the subject based on a result of the comparison.

16. The medical image diagnosis apparatus of claim 15, further comprising a notification unit configured to, when the operation for examining the subject is stopped, provide information that recommends measures to improve quality of the electrocardiogram signal.

17. The medical image diagnosis apparatus of claim 3, wherein
the biological signal measuring apparatus is a breathing sensor, and
when acquiring respiratory cycle information indicating cycle of a respiratory signal measured by the breathing sensor as the biological signal or respiratory amplitude information indicating amplitude of the respiratory signal as the information related to the measurement environment, the processing circuitry controls the operation for examining the subject based on the information related to the measurement environment.

18. The medical image diagnosis apparatus of claim 17, wherein
the processing circuitry includes a storage configured to store a respiratory cycle threshold, which is a threshold related to the cycle, or a respiratory amplitude threshold, which is a threshold related to the amplitude, in advance, and
the processing circuitry is further configured to:
compare the respiratory signal with the respiratory cycle threshold or the respiratory amplitude threshold; and
stop the operation for examining the subject based on a result of the comparison.

19. The medical image diagnosis apparatus of claim 18, further comprising a notification unit configured to, when the operation for examining the subject is stopped, provide information that recommends measures to improve quality of the respiratory signal.

20. A biological signal measuring apparatus configured to measure a biological signal of a subject, comprising processing circuitry configured to:
acquire information related to measurement environment of the biological signal, and
send the information related to the measurement environment to a medical image diagnosis apparatus.
